(19) United States
(12) Patent Application Publication
(10) Pub. No.: US 2002/0142346 A1
(43) Pub. Date: Oct. 3, 2002

(54) Binding compounds and methods for identifying binding compounds

(76) Inventors: John J. Nestor JR., Bedford, MA (US); Carol J. Wilson, Somerville, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/813,448

(22) Filed: Mar. 20, 2001

Related U.S. Application Data
(60) Provisional application No. 60/190,946, filed on Mar. 21, 2000.
(60) Provisional application No. 60/190,996, filed on Mar. 21, 2000.
(60) Provisional application No. 60/191,299, filed on Mar. 21, 2000.

(51) Int. Cl. C12Q 1/70; G01N 33/53; G01N 33/543

(52) U.S. Cl. 435/7.1; 435/5; 436/518

(57) ABSTRACT

Compositions of binding compounds for G-protein-coupled receptors and methods for identifying binding compounds for G-protein-coupled receptors are provided. Also provided are therapeutic agents comprising such compounds.

### RELATED APPLICATIONS

This application claims the benefit of U.S. Serial No. 60/190,946, U.S. Serial No. 60/190,996 and U.S. Serial No. 60/191,299, the disclosure of each of which is incorporated by reference herein. Reference is also made to the applications filed on Mar. 20, 2001 identified by attorney docket nos. CNS-005 and CNS-007, the disclosure of each of which is incorporated by reference herein.

### FIELD OF THE INVENTION

The invention relates generally to G-protein-coupled receptors (“GPCRs”), and more particularly, to binding compounds for G-protein-coupled receptors. Methods of the invention are useful for the treatment of disease by identifying and preparing therapeutic binding compounds for G-protein-coupled receptors.

### BACKGROUND OF THE INVENTION

Receptors, in general, are molecular structures located in the cell membrane or within a cell that form a weak, reversible bond with an extracellular agent such as an antigen, hormone or neurotransmitter. Each receptor is designed to bind with a specific agent. A specific family of receptors is the seven transmembrane (“7TM”), G-Protein-Coupled Receptor (“GPCR”). These receptors link with a Guanine Nucleotide-Binding Protein (“G-protein”) in order to pass on a signal from the extracellular agent with which the receptor has bound. When the G-protein binds with Guanine DiPhosphate (“GDP”), the G-protein is inactive, or in an “off position.” Likewise, when the G-protein binds with Guanine TriPhosphate (“GTP”), the G-protein is active, or in an “on position” whereby activation of a biological response in a cell is mediated.

In general, the activity of almost every cell in the body is regulated by extracellular signals. A number of physiological events in humans as well as with a wide range of organisms use protein mediated transmembrane signaling via GPCRs. Signals from a specific GPCR cause activation of a G-protein in the cell. The majority of signals are transmitted by means of GPCRs into the cell interior. The transmission of the signal is accomplished by way of ligand binding and activation of a linked G-protein. There are many varying aspects of this signaling process involving multiple receptor subtypes for GPCRs and their G-protein linked counterparts as well as a variety of linked intracellular secondary messengers. The signal transduction may result in an overall or partial activation or inactivation of an intracellular process or processes depending upon the proteins that are involved. Important signaling molecules or neurotransmitters which bind to GPCRs include, for example, morphine, dopamine, histamine, 5-hydroxytrytamine, and adenosine.

GPCRs constitute a superfamily of proteins. There are currently over 2000 GPCRs reported in literature, which are divided into three families: rhodopsin-like family, metabotropic glutamate family, and the calcitonin receptors. See e.g., Ji TH, et. al., J. Biol. Chem. 273(28): 17299-302 (1998). The reported GPCRs include both characterized receptors and orphan receptors for which ligands have not yet been identified. See e.g., Wilson S, et. al., G protein-coupled receptors. Haga T, Bernstein G, eds. CRC press, Boca Raton, pp. 97-116 (1999); Wilson S, et. al., Br. J. Pharmacol. 125(7):1387-92 (1998); and Marchese A, et. al., Trends Pharmacol. Sci. 20(9):370-375 (1999). Despite the large number of GPCRs, generally, each GPCR share a similar molecular structure. Each GPCR comprises a string of amino acid residues of various lengths. GPCRs lie within the cell membrane in seven distinct coils called transmembranes. The amino terminus of the GPCR lies outside the cell with the extracellular loops, while the carboxy-terminus lies inside the cell with the intracellular loops.

In general, the similarity of tertiary structure is shared by the G-proteins as well. Typically, G-proteins, also called heterotrimeric G-proteins, are composed of three subunits, the alpha, beta and gamma. In a typical G protein, the alpha subunit comprises two domains, a GTPase domain and an alpha-helical domain. The GTPase domain comprises helices that surround a beta sheet. The alpha-helical domain is unique to the G-proteins and comprises a long central helix surrounded by five shorter helices. The beta and gamma subunits are often referred to as the beta-gamma dimer. The beta subunit is a “beta propeller” protein comprising sheets arranged like blades on a propeller, an alpha helix and a loop that connects the helix with the “propeller blades”. The gamma subunit, on the other hand, generally, does not have an intrinsic tertiary structure; instead, it is believed to rely on the beta subunit for structural support. Interactions with the beta subunit are believed to be mediated in part by a coiled-coil interaction between the N terminal helices of the respective subunits.

The ligands for GPCRs comprise small molecules as well as peptides and small proteins. The interactions between these ligands and their receptors vary from system to system but they all may require the interaction with residues in several of the four extracellular domains and the N-terminus. GPCRs with known ligands have been associated with many diseases including multiple sclerosis, diabetes, rheumatoid arthritis, asthma, allergies, inflammatory bowel disease, several cancers, thyroid disorders, heart disease, retinitis pigmentosa, obesity, neurological disorders, osteoporosis, Human Immunodeficiency Virus (“HIV”) infection and Acquired Immune Deficiency Syndrome (“AIDS”). See e.g., Murphy P H, et. al., Pharm. Rev. 52(1):145-176 (2000); Mannstadt M, et. al., Am. J. Physiol. 277(5):F665-75 (1999); Berger E A, et. al., Ann. Rev. Immunol. 17:657-700 (1999); Saunders J, et. al., Drug Discov. Today 4(2):80-92 (1999); Hebert T E, et. al., Biochem. Cell. Biol. 76(1):1-11 (1998); Jacobson E D, et. al., Dig. Dis. 15(4-5):207-42 (1997); Meij J T, Mol. Cell. Biochem. 157(1-2):31-8 (1996); and Chanmers J, et. al., Nature 400:261-4 (1999). In general, G-protein-coupled receptors can be further divided into different classes based on the type of ligands bound: peptide, biogenic amine, nucleotide-related, lipid-based, amino acid-based, and retinal (i.e., light-based). Particular interest is the receptor class that binds peptide substrates such as, for example, CC chemokine receptor 5 and CXC chemokine receptor 4, which is the largest defined class.

While it has been demonstrated that GPCRs serve as receptors for signal transduction, it has been difficult for researchers to obtain high resolution X-ray crystallographic structures of a GPCR because of difficulties in crystallizing a 7TM protein which requires complex interactions with lipids for its native conformation. The requirement of the interaction with lipids also makes difficult the preparation of biologically active forms of GPCRs, because, in the absence of those lipids, they readily form denatured aggregates with minimal to no ability to specifically bind ligands unless great care is taken to preserve the biologically active conformation during solubilization. In the absence of an X-ray structure, a variety of approaches have been used to define the regions of GPCR that are involved in ligand binding. These approaches generally involve comparing results with non-human homologues, chimeric receptors, and point mutants to study the structural requirements for the activity of GPCRs.

Specific GPCRs involved in HIV infection and AIDS, such as, for example, CC Chemokine Receptor 5 (“CCR5”) and CXC Chemokine Receptor 4 (“CXCR4”) serve as co-receptors for HIV, such that they interact with HIV to facilitate viral entry into cells. Research, therefore, has sought to identify natural ligands acting as therapeutic agents to inhibit viral entry into cells. As with other GPCRs, CCR5 and CXCR4 are very difficult to solubilize and purify because they normally need to fold and be maintained in the presence of the native lipids of the cell membrane.

Accordingly, there is a need in the art for methods of identifying GPCR binding compounds and the identification of GPCR binding therapeutics with which to prevent or treat diseases and disorders, such as, for example, HIV infection and AIDS. Such therapeutics may comprise peptides, peptidomimetics, or small molecules that can inhibit natural ligand binding to GPCRs. Such methods and compositions are provided herein.

### SUMMARY OF THE INVENTION

The present invention provides binding compounds for GPCR and methods for identifying those binding compounds. In one embodiment, screening methods are provided to identify binding motifs for GPCR, as well as ligands capable of binding to a GPCR. In another embodiment, the invention comprises the design and identification of therapeutic peptides, peptidomimetics, or small molecules suitable for use in the prevention or treatment of diseases and disorders such as, for example, HIV infection and AIDS.

In one embodiment, methods of the invention provide for the synthesis and purification of linear and cyclic peptide libraries useful for screening and identifying a binding motif for a GPCR, as well as screening for potential ligands thereof. Methods of the invention provide for the incorporation of unnatural amino acids and amino acids of the D configuration into linear or cyclic peptides for use in such libraries. Libraries comprising peptides having such amino acids demonstrate enhanced binding affinity and duration of action in vivo resulting from resistance to proteolysis.

In a preferred embodiment, the invention provides for the use of highly diverse libraries of peptide (linear and cyclic, natural and unnatural amino acids), peptidomimetic, and small molecule compounds for the lead ligand identification step. Such ligands may be directly or indirectly agonistic or antagonistic to GPCR binding activity.

In a preferred embodiment, the invention provides for the use of phage display methods for the identification of preliminary motif information, followed by additional rounds of affinity purification with purified receptor preparations of the invention and highly diverse libraries. In a particularly preferred embodiment, phage display technology is combined with the use of cyclic peptide and/or peptidomimetic libraries.

In another embodiment of the invention, computer-aided design technology is used to virtually screen, identify, design, or validate lead compounds for agonistic or antagonistic potential with regard to GPCR activity. Such technology uses computer-generated, three-dimensional images based upon molecular and structural information of both the GPCR and the potential binding partners by virtually aligning the protein with the binding partners. In the case of a library designed for computer-aided screening, a great deal of the information necessary for lead optimization is obtained directly from the library design. In one embodiment, potential leads are identified by prior screening of an actual library or through some other means. One embodiment of the invention involves the screening of biologically appropriate drugs that relies on structure based rational drug design. In such cases, a three dimensional structure of the protein (or similar family member), peptide or molecule is determined and potential agonists and/or antagonists are designed with the aid of computer modeling. In a preferred embodiment of the invention, after an appropriate drug is identified, the drug is contacted with a GPCR, whereby a binding complex is formed between the potential drug and a GPCR. Methods of contacting the drug to a GPCR are generally understood by anyone having skill in the art of drug development.

In another embodiment, the present invention provides for the use of partially purified GPCR as the agent for carrying out the selection, identification, and improvement of tight binding ligands in identifying therapeutically useful compounds. In a preferred embodiment, the invention comprises the use of tagging methods to generate a modified GPCR that functions to facilitate purification and identification steps involved in the screening methods. In another embodiment, the invention comprises a nucleic acid sequence corresponding to GPCR fused to tag sequences (i.e., GST, FLAG, 6xHis, dual tagged with FLAG-GST, C-MYC, MBP, V5, Xpress, CBP, HA) with appropriate specific protease sites engineered into the vector.

In a particularly preferred embodiment, methods of the invention provide for solubilization and immobilization of GPCR to facilitate ligand selection methods provided herein. GPCR may be derived from any source, including without limitation: inactive, precipitated protein preparations; cell membrane preparations; and, whole cell preparations. In one embodiment, the invention provides for a method of screening combinatorial libraries directly for general affinity determination using membranes from baculovirus expression systems or any other appropriate expression system. In one embodiment of the invention, partially purified GPCR is used in carrying out the selection, identification, and improvement of tight binding ligands. In a preferred embodiment, partially purified, tagged GPCR is used in a sequestered form to screen diverse libraries (focused or highly diverse) for the affinity purification of a tight binding ligand. In a preferred embodiment of the invention, the conditions for solubilization and immobilization of the appropriate ligand provide for the use of low salt, such as, for example, low magnesium or calcium concentrations; and no sodium chloride (“NaCl”) (0.0 nM NaCl).

In another embodiment, the invention comprises the step of eluting bound components of the libraries from the immobilized protein with specific N-terminally blocked peptides or other non-sequencable analogs. In yet another embodiment, the invention comprises the step of binding combinatorial libraries to a resin-immobilized protein. In another embodiment, the invention comprises a purified polypeptide with tag sequences, which may be immobilized onto an appropriate affinity resin for assay. A further embodiment comprises the step of releasing or eluting tagged protein with its bound library with specific N-terminally blocked peptides or other non-sequencable analogs. In yet another embodiment, a method of the invention comprises the step of cleaving a tag from a protein of interest using a specific protease (as designed into the protein/vector) after immobilization onto an affinity resin and after the combinatorial library is bound to release the complex.

In yet another embodiment, the target ligand is selected from a linear peptide library, a peptidomimetic library, a cyclic peptide library, or a focused library developed using an initial motif identified by phage display techniques or a library combining any of the foregoing. In another embodiment, a target ligand is eluted from the receptor preparation using a peptide or other ligand, or by using pH change or chaotropic agents, such as urea or guanidine hydrochloride, that can disrupt the hydrogen bonding structure of water and denature proteins in concentrated solutions by reducing the hydrophobic effect. Also contemplated by the invention are ligands for GPCR identified using the methods disclosed herein. In yet another embodiment of the invention, protein sequencing techniques are used for the determination of the structure of the ligand identified by the affinity purification step.

In another embodiment, the invention comprises therapeutic agents, such as, for example, a small molecule antagonist of GPCR binding that are identified using methods of the invention appropriate for the treatment of a disease or disorder, such as, for example, HIV infection or AIDS. In another embodiment, a patient with a disease or disorder is treated with a therapeutic agent comprising a compound identified using methods of the invention, such as, for example, a small molecule antagonist of GPCR binding. In another embodiment, a patient with a disease or disorder such as, for example, HIV infection and AIDS is treated through the use of combinations of therapeutics that include, for example, GPCR inhibitors and reverse transcriptase and protease inhibitors.

A detailed description of certain preferred embodiments of the invention is provided below. Other embodiments of the invention are apparent upon review of the detailed description that follows.

### DESCRIPTION OF THE DRAWINGS

FIG. 1 shows a peptide library with a fixed, non-degenerate lysine or arginine and eight degenerate positions consisting of eighteen amino acids in approximately equal proportion.

FIG. 2 shows a peptide library screening using binding domains.

FIG. 3 shows the immobilization of GPCRs for affinity purification from libraries.

### DETAILED DESCRIPTION OF THE INVENTION

Generally, methods of the invention provide for the determination of a binding motif for GPCR. Further, methods of the invention provide for the identification of agonists or antagonists of the interaction of GPCR with its natural ligand, thereby providing for the identification of therapeutic lead compounds. Methods for library design and synthesis, and library screening that are particularly useful in the invention are described in the following patent and patent applications, the disclosure of each of which is incorporated by reference herein: Cantley et al., U.S. Pat. No. 5,532,167; Cantley, et al., U.S. Ser. No. 08/369,643, filed Dec. 17, 1998; Cantley, et al., U.S. Ser. No. 08/438,673, filed Nov. 12,1999; Hung-Sen, et al., U.S. Ser. No. 09/086,371, filed May 28, 1998; Hung-Sen, et al., U.S. Ser. No. 08/864,392, filed Jun. 24, 1999; and Lai, et al, U.S. Ser. No. 09/387,590, filed Aug. 31, 1999.

According to the methods of the invention, a GPCR is cloned and expressed, and tested for activity. The GPCR may be tagged on the C-terminus or on the N-terminus to facilitate the determination of the character of GPCR's ligand-binding properties. Exemplary tags include, without limitation, 6xHis, FLAG, GST, V5, Xpress, c-myc, HA, CBD, and MBP. The tagged GPCR is used in screening of libraries comprising, for example, linear and/or cyclic peptides having natural and/or unnatural amino acids, peptidomimetics and/or small molecules. Such peptidomimetics and small molecules may comprise any natural or synthetic compound, composition, chemical, protein, or any combination or modification of any of the foregoing that is used to screen for binding compounds of a GPCR.

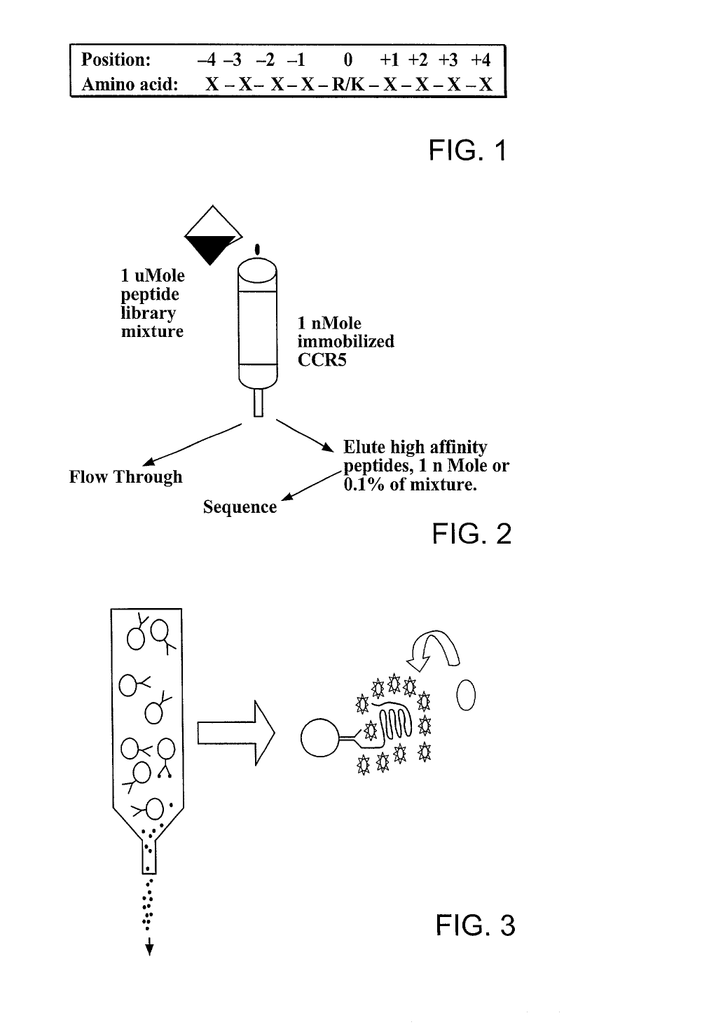

In one aspect, an oriented degenerate peptide library method useful in methods of the invention uses soluble peptide libraries consisting of one or more amino acids in non-degenerate positions, known or suspected to be important for ligand binding, and, for example, eighteen amino acids in approximately equal proportions in degenerate positions. Cysteine and tryptophan may be omitted to avoid certain analytical difficulties on sequencing. Such a library is shown in FIG. 1, where X represents a degenerate position consisting of any of eighteen amino acids and a lysine or arginine is fixed at a non-degenerate position. Additional residues can be added to the N-terminal of the sequence shown in FIG. 1 because there are often interfering substances present in the first and second sequencing cycles. Additional residues can be added at the C-terminal end to provide amino acids to better anchor the peptide to the filter in the sequencer cartridge.

Another aspect of the invention provides for the use of highly diverse libraries of peptide (linear and cyclic, natural and unnatural amino acids), peptidomimetic, and small molecule compounds for the lead identification step. For example, these ligands can be agonistic or antagonistic in their function on the receptor. Generally, the invention uses partially purified GPCR as the agent for carrying out the selection, identification, and improvement of tight binding ligands as a route to therapeutically useful compounds. In addition, the invention provides for the development and use of solubilization and immobilization procedures that facilitate efficient ligand selection methods provided herein. Specifically, optimal conditions for efficient ligand selection of certain GPCRs comprise the use of low salt, such as, for example, low or no magnesium or calcium concentrations, and no NaCl concentrations. Ligand selection methods using, for example, inactive, precipitated protein, cell membrane preparations, and whole cell preparations are further provided herein.

In one aspect of the invention, the screening step may comprise phage display technology. Such phage display systems have been used to screen peptide libraries for binding to selected target molecules and to display functional proteins with the potential of screening these proteins for desired properties. More recent improvements of the display approach have made it possible to express enzymes as well as antibody fragments on the bacteriophage surface thus allowing for selection of specific properties by selecting with specific ligands. See e.g., Smith S F, et al, Methods Enzym. 217:228-257 (1993). Phage display methods may be used for the identification of preliminary motif information, and followed by additional rounds of affinity purification with purified receptor preparations of the invention and highly diverse libraries, especially cyclic peptide and peptidomimetic libraries. The phage display methods allow the identification of motifs of natural amino acids. Information derived from phage display can be taken into affinity purification methods using, for example, synthetic libraries containing novel amino acid analogs or cyclic peptides to select ligands that have enhanced pharmaceutical characteristics. The use of initial, secondary and tertiary libraries allows a more complete definition of the specificity of the binding site. Secondary libraries may be sequenced incorporating information from the initial library. With the first library, some degenerate positions may yield high preferences for specific amino acids and these may become non-degenerate positions consisting of the preferred amino acid in a second library. See e.g., Wu R, J Biol Chem 271(27):15934-41 (1996).

Alternatively, or in addition, computer-aided design technology may be used in the screening and/or designing of peptides, peptidomimetics, and small molecules. Together with information such as, for example, the crystal structure of rhodopsin (see e.g., Palczewski, et al., Science 289(5480):739-745 (2000)) along with the sequence of a GPCR, transmembrane predictions, and any structural information obtained from mutagenesis studies, computer aided design technology may virtually screen, identify, design and validate potential compounds with regards to their GPCR activity. Computer programs that may be used to aid in the design of appropriate peptides, peptidomimetics and small molecules include, for example, Dock, Frodo and Insight. An example of a method for screening of biologically appropriate drugs relies on structure based rational drug design. In such cases, a three dimensional structure of the protein, peptide or molecule is determined or modeled and potential agonists and/or antagonists are designed with the aid of computer modeling. See e.g., Butt et al., Scientific American, December 1992-1998 (1993); West et al., TIPS, 16:67-74 (1995); Dunbrack et al., Folding & Design, 2:27-42 (1997). After an appropriate drug is identified, the drug is contacted with a GPCR, wherein a binding complex forms between the potential drug and a GPCR. Methods of contacting the drug to a GPCR are generally understood by anyone having skill in the art of drug development.

The screening step may be performed in solution phase, or with GPCR immobilized on affinity columns. In addition to the immobilization of tagged GPCR using an affinity resin, other forms of sequestration can be used to perform the affinity purification of select ligands from libraries. These include, but are not limited to the following examples. The receptor and bound library components can be separated from non-bound library components using equilibrium dialysis. The tagged receptor can be bound to specific affinity membranes, which are in the form of plates or are separate. The libraries can then be incubated with the membrane and easily washed to remove non-specific binding components. Size exclusion methodology can be used to separate a purified receptor bound library complex from unbound components after pre-incubating the receptor with the library. Additionally, a micellar complex containing the receptor (which may or may not incorporate lipids as well as detergent) can be separated after binding select affinity components from a library by differential centrifugation. Generally, the high affinity ligand can be released using low pH or high salt conditions and the structure identified by sequencing as described herein.

In order to determine those ligands that have the highest affinity to the target receptor, generally, over 200 peptide libraries may be screened to determine each library's respective inhibition binding. In general, a greater than 10% inhibition at 100 μM would be significant for continued evaluation of the sequence via affinity purification. In additional aspects of the invention, once preferred amino acids residues are identified due to high preference values by GPCR at the degenerate positions of the library, specific peptides are synthesized by the same methods as employed for library synthesis. In one embodiment of the invention, a high preference value is greater than 1. The value is determined by subtracting the control value from the sample value and dividing by the reference value. In a preferred embodiment of the invention, the preference value is greater than 1.2. In a highly preferred embodiment of the invention, the preference value is greater than 2. After synthesis of the identified peptide sequence, the peptide is purified by, for example, High Performance Liquid Chromatography (“HPLC”) and compositions are confirmed by Matrix-Assisted Laser Desorption Ionization-Time of Flight Mass Spectrometer (“MALDI-TOF MS”) and Edman Sequencing. Generally, relative affinities may be measured by modifying the radiolabel binding assay used in receptor purification.

To enhance the specificity of the motif obtained from the affinity purified peptides, other methods can be used. The bound components of the libraries can be eluted from the immobilized protein with specific N-terminally blocked peptides or other non-sequencable analogs. To avoid the release of minor contaminants from the affinity resin after binding of the library, the release/elution of the tagged GPCR with its bound library can be accomplished using specific N-terminally blocked peptides or other non-sequencable analogs. This can be done using acetylated FLAG peptide to elute GPCR-FLAG receptor from the resin. Alternatively, the tag from GPCR may be cleaved using a specific protease (as designed into the protein/vector; either enterokinase or thrombin) after immobilization onto the affinity resin and after the combinatorial library is bound to release the complex. Finally, libraries can be prescreened for their ability to bind to the receptor (using significantly less protein) by a binding assay using GPCR-containing membranes from, for example, Sf9 (Spodoptera frugiperda) or High Five (Trichoplusia ni) cells (both of which may be obtained from Invitrogen) in a single assay or in an array assay. This screening may be performed using a GPCR and a number of linear libraries and cyclic libraries to determine their effectiveness in inhibiting the natural ligand to bind.

Furthermore, in addition to GPCRs, other receptors as well as soluble proteins can be screened by this method to develop agonists or antagonists for therapeutic use. Examples of the types of proteins amenable to this screening methodology include without limitation, single transmembrane, PIG-tailed receptors, progesterone receptors, arrestins, nuclear receptors, cytokine receptors, and essentially any protein- or peptide-binding protein. IL-12 is one type of cytokine receptor that is useful in methods of the invention to identify therapeutic compounds for the treatment of diseases such as Type I diabetes. See e.g., Falcone M, et. al., Curr. Opin. Immunol. 11:670-676 (1999). PIG-tailed receptors include cathepsin D (25) and natural killer cell receptors such as CD48 and CD55 (26). See e.g., Ogier-Denis E, et. al, Biochem. Biophys. Res. Comm. 211(3):935-42 (1995); and Schubert J, et. al, Blood 76(6):1181-7 (1990). The progesterone receptor is a well-known nuclear receptor with biological relevance. See e.g., Chauchereau A, et. al., J. Biol. Chem. 275(12):8540-8548 (2000); Fewings PE, et. al., J. Neurosurg 92(3):401-405 (2000). Arrestins are a family of soluble protein-binding proteins which are implicated in a wide range of diseases because of their role in signal termination. See e.g., Wilson C J, et. al., Curr. Biol. 3(10):683-6 (1993). These are a few examples of proteins that could be easily adapted to this format.

Methods of the invention further comprise the design of therapeutic agents comprising peptides, peptidomimetics, and/or small molecules that are antagonistic to GPCR activity appropriate for the treatment of patients with a disease or disorder such as, for example, HIV infection and AIDS. Binding compounds for GPCR and the identification of optimal synthesis and purification thereof provides for an effective treatment of many diseases and disorders such as, for example, HIV infection and AIDS. Generally, small peptide ligand binding compounds of the invention, both cyclic and linear peptide ligands, demonstrate enhanced binding affinity and action, and are resistant to proteolysis. For example, peptides from libraries for CXCR4, CCR5, gonadotropin releasing hormone receptor (“GnRHR”) and certain cyclic peptides have been identified that demonstrate enhanced binding affinity to the target receptors.

Certain embodiments of the invention are described in the following examples, which are not meant to be limiting.

### EXAMPLES

### Example 1

### Preparation of Tagged GPCR and Screening Using Oriented Linear Peptide Libraries

Various GPCR vectors can be prepared for the baculovirus expression system containing epitope tags using standard techniques known by those skilled in the art that allowed for easier purification of the receptor. Tags may be incorporated at the N- or C-terminus of proteins. For GPCRs, tags at the C-terminus of the receptor can be incorporated to determine the character of the receptor's ligand-binding properties that are in the N-terminal region of the molecule. The construction of a C-terminal 6xHis tagged and C-terminal FLAG construct are given below as examples. Alternative tags may include, for example, GST, V5, Xpress, c-myc, HA, CBD, and MBP. These constructs can be made by analogous procedures using standard techniques known by those skilled in the art.

The 6xHis tag enables a one-step purification using nickel chelation. The cDNA for a GPCR can be isolated from an appropriate CDNA library using Polymerase Chain Reaction (“PCR”) and primers for the 3′ and 5′ ends of the desired gene, as well as the middle of the gene. To create a C-terminal tag, the gene of interest is subcloned into an E. coli vector, pET30a, with a C-terminal 6xHis tag. The newly created receptor is then excised and ligated into pBlueBac, a baculovirus transfer vector (Invitrogen, Carlsbad, Calif.). The construct is analyzed using both restriction digestion and sequencing, and then transfected into Sf9 insect cells (Pharmingen, San Diego, Calif.) for expression as typically done by those skilled in the art of protein expression.

A C-terminal bacterial FLAG construct can be obtained from Sigma (pFLAG-CTC). A similar strategy using standard techniques can be employed for the construction of this vector. GPCR is subcloned into the pFLAG-CTC plasmid, then excised with the C-terminal FLAG tag and ligated into the digested pBlueBac vector. The construct is analyzed using both restriction digestion and sequencing, and transfected into Sf9 or High Five insect cells for expression.

To express the GPCR gene in Sf9 or High Five cells, the pBlueBac vector containing the GPCR insert can be cotransfected with Bac-N-Blue DNA using cationic liposome mediated transfection using standard techniques. The GPCR is inserted into the baculovirus genome by homologous recombination. Cells are monitored from 24 hours post-transfection to 4-5 days. After about 72 hours, the transfection supernatant is assayed for recombinant plaques using a standard plaque assay. Cells which have the recombinant virus will produce blue plaques when grown in the presence of X-gal (5-bromo-4-chloro-3-indoyl-β-D-galactoside). These plaques is then purified and the isolate verified by PCR for correctness of recombination using standard techniques. From this, a high-titer stock is generated and infection performed from this stock for expression work using standard techniques. Controls for transfection include cells only and transfer vector.

A construct for the expression of a GPCR can be made from the starting vector pBlueBac 4.5 (Invitrogen) to remove the thrombin and enterokinase cleavage sites in the previously described vectors. The GST tag is added into the multiple cloning site by using PCR to generate the GST tag, then ligating into the digested vector (SmaI/EcoRI) using standard procedures known to those skilled in the art. Thereafter, the vector is made compatible with the Gateway technology from Lifetech for ease of manipulation. This is accomplished by ligating into the SmaI site the cassette containing the recombination sites required for this technology (obtained from Lifetech). The GPCR of interest is amplified using PCR with primers to extend the gene to contain the attachment sites for recombination. Then, the PCR product is incorporated into the baculovirus vector using BP clonase (the enzyme required for homologous recombination) to make a vector for baculovirus expression containing the GPCR with a C-terminal GST tag without the enterokinase or thrombin cleavage sites. This vector is cotransfected into Sf9 cells for preparation of the virus stock necessary for expression. The virus is plaque purified, and a PCR and sequence checked clone can be used for expression of the GPCR. Time courses with this construct with three GPCRs has demonstrated that less time is required for maximal expression of the receptor and proteolysis of the protein is less. For example, for CCR5, CXCR4 and GnRHR, 24-48 hours were required for expression. Less proteolysis resulted for GnRHR and CCR5.

To express the GPCR tagged receptor in Sf9 or High Five cells, the pBlueBac vector containing the tagged insert can be cotransfected with Bac-N-Blue DNA using cationic liposome mediated transfection using standard techniques. The tagged receptor is inserted into the baculovirus genome by homologous recombination. Cells are monitored from 24 hours posttransfection to 4-5 days. After about 24-72 hours, the transfection supernatant is assayed for recombinant plaques using a standard plaque assay. Cells which have the recombinant virus produces blue plaques when grown in the presence of X-gal (5-bromo-4-chloro-3-indoyl-β-D-galactoside). These plaques are purified and the isolate verified by PCR for correctness of recombination using standard techniques. From this, a high-titer stock can be generated and infection performed from this stock for expression work using standard techniques. Controls for transfection include cells and transfer vector.

Sf9 or High Five cells can be maintained both as adherent and suspension cultures using standard techniques known to those skilled in the art. The adherent cells can be grown to confluence and passaged using the sloughing technique at a ratio of 1:5. Suspension cells can be maintained in spinner flasks with 0.1% pluronic F-68 (to minimize shearing) for 2-3 months by sub-culturing to a density of 1×10⁶ cells/ml.

A time course after infection with recombinant virus can be used to define optimal growth conditions for expression using standard techniques. Aliquots of cells from spinner flasks are taken for this time course, centrifuged at 800× g for 10 minutes at 4° C. and both supernatant and pellet assayed by SDS-PAGE/Western blot analysis. The GPCR is expected to be in the membrane fraction (pellet). All viable systems are assayed in this fashion for levels of expression. Systems are assayed for activity using a standard radioligand binding assay on a membrane preparation using the natural ligand. If the natural ligand was not known as is often the case for orphan receptors (where the activity of the receptor has not been defined), the activity can be assayed for G protein-coupled signaling activity using standard cell-based assays known to those skilled in the art.

The membrane fraction is isolated by first pelleting the whole Sf9 cells (800× g for 10 minutes at 4° C.), then resuspending the pellet in a lysis buffer with homogenization. Typical lysis buffer is around neutral pH and contains a cocktail of protease inhibitors, both of which are standard techniques for those skilled in the art. For example, serine proteases, cysteine proteases, aspartyl proteases, and metalloproteases may be used with inhibitors, such as, for example, PMSF, aprotinin, leupeptin, phenathroline, benzamidine HCl. Membranes are pelleted. The solubilization of the receptor by different detergents (such as, but not limited to, β-dodecylmaltoside, n-octyl-glucoside, CHAPS, deoxycholate, NP-40, Triton X-100, Tween-20, digitonin, Zwittergents, CYMAL, lauroylsarcosine, etc.) is compared for quantity and activity. Solubilization may also be conducted using varying NaCl concentrations. Despite conventional thinking, the step of solubilization using low salt, for example, low calcium and magnesium concentrations and substantially in the absence of NaCl may provide unexpected optimal conditions for solubilization when compared for quantity and activity. Having 0.0 nM NaCl, although counter-intuitive, has been discovered to provide optimal conditions when solubilizing and immobilizing candidates with binding properties, such as, for example, CCR5 and CXCR4. A candidate for isolation is carried through for purification as described below.

After determining an appropriate detergent for solubilization and activity, such as, for example, NP-40, the GPCR is purified from the membrane fraction. The exact purification scheme also depends on the construct chosen, which is subject to activity and ease of solubilization. For purification of the 6xHis-tagged receptor, the membrane fraction is loaded onto a Ni-NTA column (Qiagen, Valencia, Calif.) in the presence of detergent, such as, for example, NP-40, washed extensively, and eluted with imidazole. Purification of the FLAG-tagged receptor is performed using the anti-FLAG M2 affinity matrix (Sigma, St. Louis, Mo.) in the presence of detergent and eluted with glycine. The purification is performed in the presence of an appropriate detergent, such as, for example, NP-40, found for the system in the experiment described herein. Activity of the purified receptor is assessed as described herein.

Peptides is assembled on Rink amide resin (NovaBiochem, substitution level 0/0.54 mmol/g) using an Applied Biosystems 433A synthesizer via 9-fluorenylmethyloxycarbonyl/tert.-butyl (“Fmoc”/“tBu”) based methods. tBu is used for the protection of side-chains of Asp, Glu, Ser, Thr, and Tyr, tert. -butyloxycarbonyl (“Boc”) for Lys and Trp, 2,2,4,6,7-pentamethyldihydrobenzofuran-5-sulfonyl (“Pbf”) for Arg, and triphenylmethyl (“trityl”, “Trt”) for Cys, His, Asn and Gln. The scale of the synthesis is 0.20 mmol. The resin is initially washed with N-methylpyrrolidinone (“NMP”) followed by a 1×3 minutes and 1×7.6 minutes treatment of piperidine:NMP (1:4) for N^{α}-Fmoc removal. All Fmoc-amino acids are coupled with N-[(1H-benzotriazol-1-yl) (dimethylamino)methylene]-N-methylmethanaminium hexafluorophosphate N-oxide (“HBTU”) according to the manufacturer's protocol: (a) 1.0 mmol of derivatized amino acid is dissolved in 2.1 g of NMP; (b) 0.9 mmol of 0.5 M HBTU in N,N-dimethylformamide (“DMF”) is added to the amino acid cartridge and the solution is mixed for 6 minutes; (c) 1.0 mL of 2.0 M N,N-diisopropylethylamine (“DIEA”) in NMP is added to the cartridge; (d) the HBTU solution is transferred to the resin and reacted for 40 minutes at ambient temperature while mixing. The resin is filtered and rinsed six times with a total of 90 ml of NMP and the cycle is repeated. In the one pot method to construct the highly degenerate oriented peptide libraries, a batch of resin is allowed to react with mixtures of the combinatorial amino acids without any partitioning of the resin.

Adjusting the concentrations of the amino acids in the starting mixture controls the relative coupling rates, thereby ensuring equal incorporation of the amino acids in the library. The optimization of a mixture of natural Boc and Fmoc protected amino acids for the one pot synthesis has been previously described (see e.g., U.S. Pat. No. 5,225,533; Ivanetich, et. Al., Combinatorial Chemistry, vol 267, Academic Press, San Diego, Calif. USA, p 247-260 (1996); Buettner, et al., Innovations and Perspectives in Solid Phase Synthesis: Peptides, Proteins, and Nucleic Acids, Mayflower Worldwide Ltd., Birmingham, UK, p 169-174 (1994); Ostresh, et al., Biopolymers 34:1681-9 (1994); Songyang, et. al., Methods in Mol Biol 87:87-98 (1998); and Herman, et al., Molecular Diversity 2:147-155 (1996). Cleavage reactions are performed by stirring the peptidyl-resin in trifluoroacetic acid (“TFA”):H₂O:anisole:triisopropylsilane (“iPr₃SiH”) (87.5:5:5:2.5, ˜6 mL) for 3 hours at 25° C. (see e.g., Herman et al., 1996). The filtrates are collected and the resin is further washed with TFA. Cold (−78° C.) diethyl ether is added to the combined extracts and the solution is cooled to −78° C. After removing the supernatant, the obtained precipitate is washed several times with cold ether, dissolved in glacial acetic acid and lyophilized.

For cyclic peptide libraries, Fmoc-Asp(OH)-ODmab (Dmab, 4-[N-(1-(4,4-dimethyl-2,6-dioxoxcyclohexylidene)-3-methylbutyl)amino]-benzyl) is side-chain anchored to Rink amide resin followed by chain elongation as described above. Following linear assembly, removal of the Dmab and Fmoc group is accomplished by treatments with hydrazine:DMF (1:49) for 7 minutes and piperidine:NMP (1:4) for 6×3 minutes, respectively. The resin is transferred to a syringe containing a polypropylene frit for manual cyclization. On-resin head-to-tail cyclization is performed using 7-azabenzotriazol-1-yloxy)-tris(pyrrolidino)phosphonium hexafluorophosphate (“PyAOP”):DIEA (1:2, 4 equiv) in a solution containing 1% Trition X in NMP:DMF:dichloromethane, methylene chloride, DCM) (1:1:1) for 2 hours at 55° C. The unreacted linear precursor is treated with Fmoc-Nva-OH/PyAOP/DIEA (“Nva”, “norvaline”)(1:1:2, 4 equiv) in DMF for 1×18 hours and 1×3 hours. Subsequent cleavage and side-chain deprotection as described above yields a mixture containing a cyclic peptide library and the corresponding linear (uncyclized) sequences. The desired cyclic peptide library is purified to remove the linear contaminants by reversed-phase high performance liquid chromatography (“RP-HPLC”).

Peptides and peptide libraries are characterized by HPLC, MALDI-TOF MS (Louisiana State University), and Edman degradation. MALDI-TOF MS analysis is capable of detecting the presence of high-molecular weight impurities due to incomplete deprotection, deblocking, or re-alkylation. Edman degradation provides quantitative information about the amount of each amino acid in each degenerate position in a library.

The initial libraries synthesized have single, non-degenerate orienting amino acids (i.e., M-X-X-X-X-R-X-X-X-X-A, where X is a degenerate equimolar mixture of all amino acids except cysteine). Cyclic libraries (head-to-tail) are also prepared with single, non-degenerate orienting amino acids. Through the use of these initial libraries, the optimal residues at some degenerate positions become defined and secondary libraries are made fixing these positions. For example, the head to tail cyclized library cyclo(M-X-X-X-X-R-X-X-X-X-N) indicates that the −4 position (from the fixed R) should be lysine, the −2 position should be aspartate, the −1 position should be histidine, and the +3 position should be lysine so the secondary library is cyclo(M-K-X-D-H-R-X-X-K-N).

An oriented peptide library is applied to a column containing immobilized GPCR and a small fraction of high affinity peptides isolated. A schematic diagram showing the peptide library screening using binding domains can be seen in FIG. 2. After washing, bound peptides can be eluted from the column. Next, both the bound peptides and the entire library applied to the column are submitted individually to Edman degradation, to determine the distribution of amino acids as a function of position. Finally, the preferences of amino acids at the degenerate positions are determined. For example, if serine was 5% of the amino acids at position +1 in starting library but 15% of the amino acids in position +1 in the high affinity peptides, there would be a selection for serine at the +1 position. A preference value of 3 at that position would be obtained. Table 1 provides a selective review of the use of the peptide library method with binding domains.

TABLE 1
Use Of Oriented Linear Peptide Libraries To Determine Preferred
Amino Acids For Binding Domains
(residue used for orienting sequence is shown with underline)
(p = phospho-) --“pX”

| Binding Domain | Preferred Peptide | Kd (nM) |
|---|---|---|
| PDZ | KKKKETDV | 42 |
| Src | EPQpYEEIPIYLK | 80 |
| 14-3-3 | RLSHpSLP | 55.7 |
| SH2 (src) | PYEEIY | 100 |
| SH3 | PXRPXR | |
| (amphiphysin) | | |
| SHC | NPXpY | |
| Lim | GPHydGPHydY/F | |

### Example 2

### Preparation and Screening of GST Tagged CCR5

Libraries can be synthesized on an ABI 433A (Applied Biosystems, Forster City, Calif.) with 9-fluorenylmethoxycarbonyl (Fmoc) protecting groups using a Rink Amide MBHA resin (substitution: 0.54 mmol/gm). To obtain approximately equal coupling of amino acids for degenerate positions, the amounts of amino acids are adjusted empirically after considering literature values. See e.g., Hook W, et. al., Fed. Proc. 44:1323 (1985). An exemplary coupling reagent is HBTU/HOBT/DIEA, 1 equivalent per equivalent of peptide. Cleavage is affected by a cocktail (82% TFA, 5% phenol, 5% thioanisol, 2.5% 1,2-ethanedithiol, 5% water). Peptides can be precipitated from methyl tertiary butyl ether. Libraries are characterized by MALDI-TOF MS (Louisiana State University) and by amino acid sequencing.

The initial libraries use, for example, a single, non-degenerate amino acid (i.e., X-X-X-X-R-X-X-X-X). Secondary libraries can be prepared by fixing optimal residues found at some degenerate positions. For example, X-X-X-X-R-X-X-X-X may indicate that the −4 position should be proline so the secondary library would be P-X-X-X-R-X-X-X-X.

The use of peptide libraries with the GST form of the receptor is provided herein. In the case of the GST tagged GPCR, the receptor can be exposed to the library and separation of free and bound peptides accomplished by pelleting the membranes by centrifugation. The GST-tagged purified receptor is incubated with a peptide library, about 1 μmole of peptide and about 1 nmole of binding sites. After incubation, receptor with bound peptide is separated from unbound peptides by centrifugation (receptor.peptide complex in the pellet, unbound peptide in the supernatant). Nonspecifically bound peptides can be removed by exhaustive washing, and resuspension of the pellet in low pH (≦2.5) is used to remove the bound peptide. This peptide is sequenced to determine the consensus sequence.

When a FLAG-tagged GPCR is used in peptide library screening, the receptor may be immobilized on an anti-FLAG M2 affinity matrix (St. Louis, Mo.). An additional purification approach may use GST constructs and immobilized glutathione (Pierce, Rockford, Ill.).

Both the bound peptide mixture and the starting peptide library can be sequenced using standard techniques. The amounts of each amino acid, as a function of position, is determined. Preference values for each amino acid at each position is calculated by comparing the amounts of amino acids present in the starting library and bound fraction of peptides. These procedures are used to generate preferred sequences of peptides interacting with many binding domains and are described in Table 1.

Also, secondary libraries can be sequenced incorporating information from the initial library. For the first round of characterization, phage display technology is also used to identify preliminary binding motifs. The phage display method provides for the identification of motifs of natural amino acids. Phage display technology involves the insertion of DNA sequences into a gene coding for one of the phage coat proteins. The gene is inserted in a particular location so that the expressed protein insert can interact with other molecules. As a result, the encoded peptide or protein sequence will be presented on the surface of the phage and exposed for binding. By inserting degenerate nucleotides, each phage can express a different peptide sequence (“a phage library”). Incubation of this phage library with the immobilized receptor can be used to identify sequences which specifically bind to the receptor. Even weak signals can detected because they can be amplified by growing the isolated phage. Information derived from phage display is applicable to affinity purification methods using synthetic libraries containing novel amino acid analogs or cyclic peptides to select ligands that have enhanced pharmaceutical characteristics. The use of initial, secondary and tertiary libraries provided a complete definition of the specificity of the binding site.

Once preferred amino acids residues are identified using high preference values at the degenerate positions of the library, specific peptides can be synthesized by methods as employed for library synthesis. They can be purified by HPLC and compositions confirmed by MALDI-TOF MS.

Relative affinities can be measured by modifying the iodinated binding assay used in receptor purification. Therefore, the ability of these peptides to displace [¹²⁵I]-ligand from the purified receptor membranes can be measured. If, however, the natural ligand was not known, the activity can be measured using a cell-based assay standard in the art. Also, association constants can be determined using standard techniques in the art.

### Example 3

### Preparation and Analysis of Tagged CCR5, and Library Screening Thereof

A. Cloning and Expression of CCR5

CCR5 cDNA (see sequence in FIG. 4) was obtained from Receptor Biology (Beltsville, Md.) in the vector pCDNA3. Tags were added to the C-terminus of the receptor for use in immobilizing them for affinity purification assays using standard techniques, as described herein. In addition, CCR5 cDNA correlating to GenBank Accession #U57840 may be used in the vector pCDNA3. The only difference between CCR5 cDNA from Receptor Biology and CCR5 cDNA correlating to GenBank Accession #U57840 is a point mutation at base 180 that changes Leucine to a Glutamine in the amino acid sequence. The following are specific examples from experiments using this tagging method.

Construction of CCR5 with C-Terminal Histidine Tag (Insect Select Expression System)

The CCR5-HIS construct was derived from this system. PCR was performed using CCR5/pcDNA3 (from Receptor Biology) as the template and primers 5-Age His and 5-Spe His. The first primer introduced a unique Spe I site just before the initiator ATG of CCR5. The second primer mutated the stop codon of CCR5 into an Age I site in-frame with the histidine tag of pIZT/V5-His (Invitrogen, Carlsbad, Calif.). The PCR product was digested with Spe I and Age I, then ligated into similarly digested pIZT/V5-His. This construct is identified as CCR5-His-PIZT.

Construction of CCR5 with C-Terminal Histidine Tag (Baculovirus Expression System)

CCR5-His-PIZT was digested with Hae II and Spe I, then filled in with Klenow fragment. The fragment containing CCR5-His was ligated into pBluebac 4.5 (Invitrogen) that was previously digested with Nhe I and blunted with Klenow. The construct was checked for correctness of orientation. This is called CCR5-HIS. The construct was confirmed by restriction digestion and sequencing using standard techniques. This construct has been used for expression and has been determined to be expressed sufficiently and in active form for use in the affinity purification screening.

Construction of CCR5 with C-Terminal FLAG Tag:

PCR using standard techniques was performed using CCR5/pcDNA3 (from Receptor Biology) as the template and the primers 5-Xho pFLAG and 5-Sal pFLAG. The first primer engineered a unique Xho I site just before the initiator ATG of CCR5. The second primer mutated the stop codon of CCR5 into a Sal I restriction site (in-frame with the FLAG tag of pFLAG-CTC from Sigma). The PCR product was digested with Xho I and Sal I and ligated into similarly digested pFLAG-CTC (a bacterial expression vector). This construct is called CCR5-FLAG-CTC. CCR5-FLAG was then digested with Xho I and Sca I, and filled in with Klenow fragment. The fragment containing CCR5-FLAG-CTC was ligated into pBluebac 4.5 that was first digested with Nhe I then blunted with Klenow. This final construct is identified as CCR5-FLAG. The construct was confirmed by restriction digestion and sequencing using standard techniques. This construct has been used for expression and has been determined to be expressed sufficiently and in active form for use in affinity purification screening.

Construction of CCR5 with C-Terminal GST Tag:

PCR was performed using CCR5/pcDNA3 (from Receptor Biology) as the template and primers GST-BamH1 and GST-Nde1. The first primer mutated the stop codon of CCR5 into a BamH I restriction site (in-frame with the FLAG/GST tag of pESP-3). The second primer introduced a unique Nde I site at the initiator codon of CCR5. The PCR product was digested with BamH I and Nde I and ligated into similarly digested pESP-3 (a yeast expression vector.) This construct is called pCP8. pCP8 was then digested with Nde I and Sma I, and filled in with Klenow fragment. The fragment containing CCR5-GST was ligated into pBluebac 4.5 that was first digested with Bgl II then blunted with Klenow. This final construct is identified as pCP10. When describing the protein, the construct is identified as CCR5-GST. The construct was confirmed by restriction digestion and sequencing using standard techniques. This construct has been used for expression and has been determined to be expressed sufficiently and in active form for use in affinity purification screening.

The vectors for the three new constructs (for CCR5-FLAG, CCR5-GST, and CCR5-HIS) were used to co-transfect Sf9 cells for the production of a viral stock of each. These viral stocks were purified using a standard plaque assay and then used in experiments to infect for the optimization of expression of CCR5 with its various C-terminal tags. High Five cells (Invitrogen) were also transfected with these CCR5 tagged constructs and tested for expression of CCR5. All constructs were determined to express the appropriately tagged receptor. Expression levels after 72 hours were as much as 5 times greater in High Five cells than those for Sf9 cells. All of the above described experiments were done using standard techniques known to those skilled in the art.

A fourth construct for the expression of CCR5 was made from the starting vector pBlueBac 4.5 (Invitrogen) to remove the thrombin and enterokinase cleavage sites in the previously described vectors. The GST tag was added into the multiple cloning site by using PCR to generate the GST tag, then ligating into the digested vector (SmaI/EcoRI) using standard procedures known to those skilled in the art. Next, the vector was made compatible with the Gateway technology from Lifetech for ease of manipulation. This was done by ligating into the SmaI site the cassette containing the recombination sites required for this technology (from Lifetech). CCR5 was amplified using PCR with primers to extend the gene to contain the attachment sites for recombination. Then, the PCR product was incorporated into the baculovirus vector using BP clonase (the enzyme required for homologous recombination) to make a vector for baculovirus expression containing CCR5 with a C-terminal GST tag without the enterokinase or thrombin cleavage sites. This vector was cotransfected into Sf9 cells for preparation of the virus stock necessary for expression. The virus was plaque purified, and a PCR and sequence checked clone was used for expression of CCR5. A time course with this construct showed that less proteolysis of the protein was observed and less time was necessary to obtain maximal expression of the receptor.

B. Activity of CCR5

Each of the tagged CCR5 genes (CCR5-GST, CCR5-FLAG, and CCR5-HIS) were expressed in Sf9 and High Five cells, as described in herein. Whole cells from Sf9 and High Five cell lines were lysed using hypotonic buffers (10 mM Tris, pH 7.4, 5 mM EDTA), and membrane preparations were made by homogenization and centrifugation using standard techniques known to those skilled in the art. Membrane preparations for CCR5-GST, CCR5-FLAG, and CCR5-HIS were assayed using a standard radioligand binding assay respectively. Binding assays were performed with 5 μg of membranes in 50 mM Hepes, pH 7.5, 1% BSA. The radioligand MIP1-β (obtained from New England Nuclear, “NEN”) was incubated with membranes at room temperature for 1 hour with and without cold MIP 1 a (a competing ligand; natural ligands for CCR5 are RANTES, MIP1β, and MIP1α), filtered, washed, and radioactive counts bound were detected using scintillation counting. Uninfected cells were used as a control for this experiment. The activity of the membrane preparations was comparable to that obtained by Receptor Biology (K_{d}<1 nM for MIP1-β binding) at least having 20% active protein.

C. Solubilization of CCR5

Both lysed whole cells and membrane preparations have been used for solubilization. Solubilization of the tagged versions of CCR5 (CCR5-FLAG, CCR5-GST, and CCR5-HIS) have been performed using many different combinations of detergents (such as, for example, NP-40, Triton X-100, β-D-maltoside, n-octylglucoside, CYMAL, Zwittergents, Tween-20, lysophosphatidyl choline, CHAPS, etc.,) salts (such as, for example, NaCl, CaCl₂, MgCl₂, MnCl₂, KCl, etc.,) buffers (such as, for example, Tris, Hepes, Hepps, Pipes, Mes, Mops, acetate, phosphate, imidazole, etc.,) at pH's ranging from about 6.8 to about 8.2. Conditions for optimal solubilization were found using Zwittergent 3-14 and low salt, e.g., low magnesium and calcium, but no NaCl (0.0 nM NaCl) at pH 8.1. In a preferred embodiment, at least 20% of the solubilized, immobilized protein is active. In highly preferred embodiments, at least 30%, 40%, 50% and 75% of the solubilized, immobilized protein is active.

D. Immobilization of CCR5

After solubilization, both CCR5-GST and CCR5-FLAG were immobilized onto affinity columns for purification and for use as active proteins for screening of peptide libraries. A schematic diagram showing the immobilization of GPCRs for affinity purification from libraries is shown in FIG. 3. CCR5-GST was bound and immobilized onto glutathione-agarose (Pierce) and glutathione-sepharose (Amersham Pharmacia Biotech) and CCR5-FLAG was immobilized onto a specific antibody column that recognizes the FLAG epitope (M2 column, Sigma). The immobilization of the protein in active form required the use of detergents and appropriate pH and salt conditions to maintain activity while on the column. This activity was determined by radioactive binding using radiolabeled MIP-1β (as with the membrane assay above) and competition with cold MIP-1α. Uninfected cells have been used as controls for this activity, as well as the column alone. These experiments demonstrated the ability to immobilize microgram quantities of the receptor in pure form (sufficient for affinity purification screening) onto resin in active form.

E. Peptide Library Synthesis

Libraries were synthesized on an ABI 433A (Applied Biosystems, Forster City, Calif.) with 9-fluorenylmethoxycarbonyl (Fmoc) protecting groups using a Rink Amide MBHA resin (substitution: 0.54 mmol/gm). When a mixture of amino acids was to be used for degenerate positions, the approximately equal coupling of amino acids was obtained by adjusting the amounts of amino acids empirically after considering literature values. See. e.g., Ivanetich et. al., Combinatorial Chemistry, vol 267, Academic Press, San Diego, Calif. USA, p 247-260 (1996). The coupling reagent was HBTU/HOBT/DIEA, 1 equivalent per equivalent of peptide. Cleavage was effected by a cocktail (82% TFA, 5% phenol, 5% thioanisol, 2.5% 1,2-ethanedithiol, 5% water). Peptides were precipitated from methyl tertiary butyl ether. Libraries were characterized by MALDI-TOF MS (Louisiana State University) and by amino acid sequencing.

The initial libraries used a single, non-degenerate basic amino acid (i.e., M-X-X-X-X-W-X-X-X-X-A-K-K-K). Through the use of these initial libraries, the optimal residues at some or all degenerate positions became defined. Secondary libraries were made if all of the positions were not defined, thereby fixing the defined positions.

F. Screening of Peptide Libraries Using Immobilized CCR5

With protein immobilized to the specific resin (for example, CCR5-GST to glutathione-sepharose or CCR5-FLAG to M2 antibody-agarose), screening of billions of compound can take place by incubating them together and allowing the natural preferences and binding affinities to purify the ligands which are preferred by the CCR5. These experiments were performed using the linear library 4P4(+) and may be performed with other libraries.

G. Phage Display

As an alternative or additional method useful in screening, immobilized functional CCR5 was used to isolate phage that bind to CCR5 using standard techniques known to those skilled in the art. Subtraction of the background from the glutathione-sepharose beads, BSA, and MIP1β used in the assay was performed by incubation of the phage library with the mixture of these components. After incubating subtracted phage libraries (i.e., NEB PhD C7C) with the receptor, bound phage were eluted both with the natural CCR5 ligand (MIP-1β) and with glycine, pH 2.2. PhD C7C is a particular phage library with 7 random amino acids between disulfides, and can be obtained from New England Biolabs (“NEB”). Multiple rounds of screening were performed. Both conditions have provided specific sequences which bind to CCR5 and inhibit ligand binding.

### Example 4

### Preparation and Analysis of Tagged CXCR4, and Library Screening Thereof

A. Cloning and Expression of CXCR4

CXCR4 was isolated from a spleen cDNA library in two halves and spliced together. These two fragments were isolated using PCR technology and primers to the 3′ and 5′ ends and the middle of the CXCR4 gene. A full-length clone was not isolated with the 3′ and 5′ primers; however, two halves were isolated and ligated together using a unique BamH I site in the gene. The identity of the construct was confirmed by sequencing. An alternate splice shorter form was also isolated, which is called CXCR4s. Tags were added to the C-terminus of the receptor for use in immobilizing them for affinity purification assays using standard techniques. The following are specific examples from experiments using this tagging method.

Construction of CXCR4 with C-Terminal Histidine Tag (Insect Select Expression System)

A previous construct containing the gene for GNRHR (gonadotropin releasing hormone receptor) was used to make the first CXCR4 construct. The gene for GnRHR was spliced out and replaced by the isolated cDNA for CXCR4. This vector was originally the pet30a vector with the 6xHis tag at the C-terminus.

Construction of CXCR4 Construct with C-Terminal FLAG tag:

PCR was performed using the primers 5′ BspE1 CXCR4 and 3 ′ Bgl CXCR4 and engineered with unique sites for ligation of CXCR4 in frame with the FLAG tag of pFLAG-CTC (a bacterial expression vector) from Sigma. This construct is called CXCR4-FLAG-CTC. CXCR4-FLAG was then removed by digestion and filled in with Klenow fragment. The fragment containing CXCR4-FLAG was ligated into pBluebac 4.5 that was first digested then blunted with Klenow. This final construct is called CXCR4-FLAG. The construct was confirmed by restriction digestion and sequencing using standard techniques. This construct has been used for expression and has been determined to be expressed sufficiently and in active form for use in affinity purification screening.

Construction of CXCR4 Construct with C-Terminal GST tag:

The newly constructed CXCR4-FLAG cDNA was removed from the CTC vector and subcloned into another construct, CCR5-GST, in place of the CCR5 (using Bgl and BspE1). This created the vector for CXCR4GST using one step. The construct was confirmed by restriction digestion and sequencing using standard techniques. This construct has been used for expression and been determined to be expressed sufficiently and in active form for use in affinity purification screening.

Construction of CXCR4 with N-Terminal 6xHis Tag:

This construct was prepared by subcloning the CXCR4 into the commercially available vector, pBluebacHis2b (Invitrogen). The construct was confirmed by restriction digestion and sequencing using standard techniques.

The vectors for the three new constructs, CXCR4-FLAG, CXCR4-GST, and CXCR4-HIS, were used to co-transfect Sf9 cells for the production of a viral stock of each. These viral stocks were purified using a standard plaque assay and then used in experiments to infect for the optimization of expression of CXCR4 with its various C-terminal tags. High Five cells (Invitrogen) were also transfected with these CXCR4 tagged constructs and tested for expression of CXCR4. All constructs were determined to express the appropriately tagged receptor. Expression levels after 72 hours were as much as 5 times greater in High Five cells than those for Sf9 cells.

B. Activity of CXCR4

Each of the tagged CXCR4 genes (CXCR4-FLAG, CXCR4-GST, and CXCR4-HIS) were used to co-transfect Sf9 and High Five cells, as described herein. Whole cells from Sf9 and High five cell lines were lysed using hypotonic buffers (10 mM Tris, pH 7.4, 5 mM EDTA), and membrane preparations were made by homogenization and centrifugation using standard techniques known to those skilled in the art. Membrane preparations for CXCR4-GST, CXCR4-FLAG, and CXCR4-HIS were assayed using a standard radioligand binding assay. The radioligand [¹²⁵I]-SDF-1 (NEN) was incubated with membranes at room temperature for 1 hour with and without cold SDF-1 (a competing natural ligand), filtered, washed, and radioactive counts bound were detected using scintillation counting. Uninfected cells were used as a control for this experiment. The activity of the membrane preparations resulted in at least 20% active protein.

C. Solubilization of CXCR4

Both lysed whole cells and membrane preparations have been used for solubilization. Solubilization of the tagged versions of CXCR4 (CXCR4-FLAG, CXCR4-GST, and CXCR4-HIS) have been performed using many different combinations of detergents (such as, for example, NP-40, Triton X-100, β-D-maltoside, n-octylglucoside, CYMAL, Zwittergents, Tween-20, lysophosphatidyl choline, CHAPS, etc.,) salts (such as, for example, NaCl, CaCl₂, MgCl₂, MnCl₂, KCl, etc.,) buffers (such as, for example, Tris, Hepes, Hepps, Pipes, Mes, Mops, acetate, phosphate, imidazole, etc.,) at pH's ranging from about 6.8 to about 8.2. Conditions for optimal solubilization were found using Zwittergent 3-14 and low salt, e.g., low magnesium and calcium, but no NaCl (0.0 nM NaCl) at pH 8.1. In a preferred embodiment, at least 20% of the solubilized, immobilized protein is active. In highly preferred embodiments, at least 30%, 40%, 50% and 75% of the solubilized, immobilized protein is active.

D. Immobilization of CXCR4

After solubilization, CXCR4-GST was immobilized onto affinity columns for purification and as active protein ready for use in screening of peptide libraries. A schematic diagram showing the immobilization of GPCRs for affinity purification from libraries is shown in FIG. 3. CXCR4-GST was bound and immobilized onto glutathione-agarose (Pierce) and glutathione-sepharose (Amersham Pharmacia Biotech). The immobilization of the protein in active form required the use of detergents and appropriate pH and salt conditions to maintain activity while on the column. This activity was determined by radioactive binding using radiolabeled SDF-1 (as with the membrane assay above) and competition with cold SDF-1. Uninfected cells was used as controls for this activity, as well as the column alone. These experiments demonstrated the ability to immobilize microgram quantities of the receptor in pure form sufficient for affinity purification screening onto resin in active form.

E. Peptide Library Synthesis

Libraries were synthesized on an ABI 433A (Applied Biosystems, Forster City, Calif.) with 9-fluorenylmethoxycarbonyl (Fmoc) protecting groups using a Rink Amide MBHA resin (substitution: 0.54 mmol/gm). When a mixture of amino acids was used for degenerate positions, the approximately equal coupling of amino acids was obtained by adjusting the amounts of amino acids empirically after considering literature values. See. e.g. Ivanetich et. al., Combinatorial Chemistry, vol 267, Academic Press, San Diego, Calif. USA, p 247-260 (1996). The coupling reagent was HBTU/HOBT/DIEA, 1 equivalent per equivalent of peptide. Cleavage was effected by a cocktail (82% TFA, 5% phenol, 5% thioanisol, 2.5% 1,2-ethanedithiol, 5% water). Peptides were precipitated from methyl tertiary butyl ether. Libraries were characterized by MALDI-TOF MS (Louisiana State University) and by amino acid sequencing.

The initial libraries used a single, non-degenerate basic amino acid (i.e., M-X-X-X-X-W-X-X-X-X-A-K-K-K). Through the use of these initial libraries, the optimal residues at some or all degenerate positions became defined. Secondary libraries were made if not all of the positions were defined, fixing the defined positions.

F. Screening of Peptide Libraries Using Immobilized CXCR4

With protein immobilized to the specific resin (for example, CXCR4-GST to glutathione-sepharose), screening of billions of compound can take place by simply incubating them together and allowing the natural preferences and binding affinities to purify the ligands which are preferred by CXCR4. These experiments have been performed with ten libraries and can be performed with other libraries.

G. Phage Display

As an alternative or additional method useful in screening, immobilized functional CXCR4 was used to isolate phage which bind to CXCR4 using standard techniques known to those skilled in the art. Subtraction of the background from the glutathione-sepharose beads, BSA, and SDF1α used in the assay was performed by incubation of the phage library with the mixture of these components. After incubating subtracted phage libraries (i.e., NEB PhD C7C) with the receptor, bound phage were eluted both with the natural CXCR4 ligand (SDF1α) and with glycine, pH 2.2. PhD C7C is a particular phage library with 7 random amino acids between disulfides, and can be obtained from New England Biolabs (“NEB”). Multiple rounds of screening were performed. Both conditions have provided specific sequences which bind to CXCR4 and inhibit ligand binding.

### Example 5

### Prevention or Treatment of Diseases and Disorders

Presently, certain complications, however, are encountered during the production, formulation and use of therapeutic peptides, peptidomimetic, or small molecule antagonists and agonists of GPCR binding used for the prevention and treatment of diseases and disorders, such as, AIDS and HIV infection. Biologically appropriate antagonists or agonists that minimize the cost and technical difficulty of commercial production of therapeutic binding compounds of a GPCR are further contemplated by the present invention. In addition, biologically appropriate antagonists or agonists of GPCR binding that do not confer an immunilogical response to the antagonist or agonist such that it interferes with the effectiveness thereof are contemplated by the invention. Moreover, appropriate formulations that confer commercially reasonable shelf life of the produced antagonist or agonist of GPCR binding, without significant loss of biological efficacy are contemplated in the present invention. Furthermore, useful dosages for administration to an individual are contemplated in the present invention appropriate for the prevention and treatment of diseases and disorders, such as AIDS and HIV infection.

The identification of appropriate candidates that, alone or admixed with other suitable molecules, that are competent in inhibiting GPCR binding are contemplated by the invention. Such candidates further contemplate the production of commercially significant quantities of the aforementioned identified candidates that are biologically appropriate for the prevention and treatment of diseases and disorders such as AIDS and HIV infection. Moreover, the invention provides for the production of therapeutic grade commercially significant quantities of a GPCR binding antagonists, agonists or derivatives in which any undesirable properties of the initially identified analog, such as in vivo toxicity or a tendency to degrade upon storage, are mitigated.

Methods of preventing and treating disease, such as AIDS and HIV infection also, after the identification and design of a peptide, peptidomimetic, or small molecule antagonist of GPCR binding activity, comprise the step of administering a composition comprising such a compound capable of inhibiting GPCR binding as described herein. Administration may be by any compatible route. Thus, as appropriate, administration may include oral or parenteral, including intravenous and intraperitoneal routes of administration. A particularly preferred method is by controlled-release injection of a suitable formulation. In addition, administration may be by periodic injections of a bolus of a composition, or may be made more continuous by intravenous or intraperitoneal administration from a reservoir that is external (e.g., an intravenous bag) or internal (e.g., a bioerodable implant).

Therapeutic compositions contemplated by the present invention may be provided to an individual by any suitable means, directly (e.g., locally, as by injection, implantation or topical administration to a tissue locus) or systemically (e.g., parenterally or orally). Where the composition is to be provided parenterally, such as by intravenous, subcutaneous, intramolecular, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the composition may comprise part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance.

Useful solutions for parenteral administration may be prepared by any of the methods well known in the pharmaceutical art, described, for example, in REMINGTON'S PHARMACEUTICAL SCIENCES (Gennaro, A., ed.), Mack Pub., 1990. Formulations of the therapeutic agents of the invention may include, for example, polyalkylene glycols such as polyethylene glycol, oils of vegetable origin, hydrogenated naphthalenes, and the like. Formulations for direct administration, in particular, may include glycerol and other compositions of high viscosity to help maintain the agent at the desired locus. Biocompatible, preferably bioresorbable, polymers, including, for example, hyaluronic acid, collagen, tricalcium phosphate, polybutyrate, lactide, and glycolide polymers and lactide/glycolide copolymers, may be useful excipients to control the release of the agent in vivo. The concept of a controlled release injectable formulation for peptide drugs is well-accepted and offers several advantages. First, for example, bioavailabilities are high. Second, treatment regimens can consist of once per month or per three months (like Abbott's Leupron®), or once per year (e.g. Alza's Viadur®). Third, controlled release injectable formulations substantially reduces the doses that can be used (the Leupron injection dose is 1 mg/day but the 90 day formulation uses is 11.25 mg total). Also, increased efficacy can be achieved if the therapeutic is present continuously to prevent infectivity. This consideration is particularly important in view of the need to approach a cure for this disease by preventing the reformation of slow-to-clear deposits of infection such as the memory T cell compartment. See e.g., Lee, V., ed. Peptide and Protein Drug Delivery. Marcel Dekker, Inc., NY (1991).

Other potentially useful parenteral delivery systems for these agents include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or cutric acid for vaginal administration.

Additional aspects and embodiments of the invention are apparent to the skilled artisan.

We claim:

1. A method of identifying a binding compound for a G-protein-coupled receptor comprising the steps of:
a) providing a library of two or more molecules;
b) providing a molecule having a binding property corresponding to a G-protein-coupled receptor, wherein said molecule is attached to a support;
c) binding a molecule from said library of two or more molecules to said molecule having a binding property corresponding to a G-protein-coupled receptor attached to said support;
d) separating said bound molecule from said molecule attached to said support; and
e) identifying said bound molecule as a binding compound for a G-protein-coupled receptor.

2. The method of claim 1, wherein said library of two or more molecules is selected from the group consisting of linear peptides, cyclic peptides, natural amino acids, unnatural amino acids, peptidomimetic compounds and small molecule compounds.

3. The method of claim 1, wherein said molecule having a binding property corresponding to a G-protein-coupled receptor is a partially purified G-protein-coupled receptor.

4. The method of claim 1, wherein at least one of said two or more molecules is selected from a group consisting of a peptide, a peptidomimetic or small molecule that can substitute for a protein capable of binding to receptors, enzymes or other proteins.

5. The method of claim 1, further comprising the step of solubilizing said molecule having a binding property corresponding to a G-protein-coupled receptor substantially in the absence of sodium chloride.

6. The method of claim 1, further comprising the step of solubilizing said molecule having a binding property corresponding to a G-protein-coupled receptor using a buffer having a low salt concentration.

7. The method of claim 1, wherein at least one of said two or more molecules comprises a molecule having an antagonistic effect on G-protein-coupled receptor binding activity.

8. The method of claim 1, wherein said library comprises a phage library.

9. The method of claim 1, wherein said steps a, b, c, and d are repeated at least once prior to said step e.

10. The method of claim 1, wherein said molecule having a binding property corresponding to a G-protein-coupled receptor comprises a G-protein-coupled receptor molecule and a tag selected from the group consisting of GST, FLAG, 6xHis, C-MYC, MBP, V5, Xpress, CBP, and HA).

11. A binding compound for a G-protein-coupled receptor identified according to the method of claim 1.

12. A method of preventing disease or disorder in a patient, the method comprising administering to said patient a therapeutic composition comprising the compound of claim 9 in a physiological carrier.

13. The method of claim 12, wherein said disease or disorder is HIV infection.

14. The method of claim 12, wherein said disease or disorder is AIDS.

15. A method of treating or preventing disease or disorder in a patient, the method comprising administering to said patient a therapeutic composition comprising the compound of claim 9 in a controlled release injectable formulation.

16. The method of claim 15, wherein said disease or disorder is HIV infection.

17. The method of claim 15, wherein said disease or disorder is AIDS.

18. A computer-aided method for identifying relative binding affinity of a test molecule to a G-protein-coupled receptor, comprising the steps of:
a) entering input data characterizing a G-protein-coupled receptor into a computer program;
b) entering input data characterizing at least one test peptide-like molecule, each of known sequence but unknown binding affinity;
c) analyzing each applied test peptide-like molecule using the computer program to generate a prediction of a relative binding affinity for each test peptide-like molecule, and outputting such prediction.

19. A method for determining an amino acid sequence motif for an interaction site of a binding compound for a G-protein-coupled receptor, comprising the steps of:
a) contacting a peptide library with a molecule having a binding property corresponding to a G-protein-coupled receptor under conditions which allow for interaction between said molecule having a binding property corresponding to a G-protein-coupled receptor and said peptide library;
b) allowing said molecule having a binding property corresponding to a G-protein-coupled receptor to interact with said peptide library such that a complex is formed between said molecule having a binding property corresponding to a G-protein-coupled receptor and a subpopulation of library members capable of interacting with said molecule having a binding property corresponding to a G-protein-coupled receptor;
c) separating said subpopulation of library members capable of interacting with said molecule having a binding property corresponding to a G-protein-coupled receptor from library members that are incapable of interacting with said molecule having a binding property corresponding to a G-protein-coupled receptor;
d) linearizing said subpopulation of library members capable of interacting with said molecule having a binding property corresponding to a G-protein-coupled receptor;
e) determining a relative abundance of different amino acid residues at each degenerate position within a mixture of linearized library members; and
f) determining an amino acid sequence motif for an interaction site of said molecule having a binding property corresponding to a G-protein-coupled receptor, based upon said relative abundance of different amino acid residues at each degenerate position within the mixture of linearized library members.

20. An amino acid sequence motif for a binding compound for a G-protein-coupled receptor identified according to the method of claim 19.

21. A binding compound having the amino acid sequence motif for a G-protein-coupled receptor determined by the method of claim 19.

22. The method of claim 19, wherein at least one member of said peptide library comprises at least one unnatural amino acid.

23. The method of claim 19, wherein said molecule having a binding property corresponding to a G-protein-coupled receptor is selected from the group consisting of linear peptides, cyclic peptides, natural amino acids, unnatural amino acids, peptidomimetic compounds and small molecule compounds.

24. The method of claim 19, wherein said peptide library comprises at least one molecule selected from the group consisting of linear peptides, cyclic peptides, natural amino acids, unnatural amino acids, peptidomimetic compounds and small molecule compounds.

25. A library comprising members based upon an amino acid sequence motif for an interaction site of a binding compound for a G-protein-coupled receptor, the motif being determined by permitting at least one peptide member from a peptide library to interact with said binding compound for a G-protein-coupled receptor, and determining an amino acid sequence of at least one peptide that interacts with said binding compound for a G-protein-coupled receptor.

26. A method of solubilizing and immobilizing a compound corresponding to the binding property of a G-protein-coupled receptor, wherein the solubilization and immobilization is conducted substantially in the absence of sodium chloride when determining a compound corresponding to the binding of a G-protein-coupled receptor.

27. A method of solubilizing and immobilizing a compound corresponding to the binding property of a G-protein-coupled receptor, wherein the solubilization and immobilization is conducted by a using a low salt concentration when determining a compound corresponding to the binding of a G-protein-coupled receptor.

28. The method of claim 27, wherein said low salt concentration comprises a pre-determined amount of magnesium and calcium.

29. A G-protein-coupled receptor transfer vector constructed wherein said transfer vector comprises a G-protein-coupled receptor molecule and a tag selected from the group consisting of GST, FLAG, 6xHis, C-MYC, MBP, V5, Xpress, CBP, and HA.

30. A method of using three-dimensional structure of G-protein-coupled receptor in a drug screening assay comprising:
a) selecting a potential drug by performing rational drug design with the three-dimensional structure, wherein said selecting step is performed in conjunction with computer modeling;
b) contacting the potential drug with a first molecule comprising a first G-protein-coupled receptor; and
c) detecting the binding of the potential drug with said first molecule; wherein a potential drug is selected as a drug if the potential drug binds to said first molecule.

31. The method of claim 30, wherein said first molecule is labeled.

32. The method of claim 30, wherein said first molecule is bound to a solid support.
